(19) United States
(12) Patent Application Publication
(10) Pub. No.: US 2012/0289817 A1
(43) Pub. Date: Nov. 15, 2012

(54) OPTICAL RESPIRATION STATUS SENSOR

(75) Inventors: Eduardo M. Rey, Winter Springs, FL (US); Mark D. Nelson, Satellite Beach, FL (US); Harold J. Cates, Orlando, FL (US)

(73) Assignee: KONINKLIJKE PHILIPS ELECTRONICS N.V., EINDHOVEN (NL)

(21) Appl. No.: 13/510,969

(22) PCT Filed: Nov. 18, 2010
(86) PCT No.: PCT/IB2010/055272

§ 371 Date: May 21, 2012

Related U.S. Application Data
(60) Provisional application No. 61/287,001, filed on Dec. 16, 2009.

(51) Int. Cl. A61B 5/113; A61B 5/055

(52) U.S. Cl. 600/413

(57) ABSTRACT

A subject monitor includes a belt (16) that circumscribes a cyclically moving portion of a subject (14), such as a chest cavity or abdomen. A optical sensing device (18) includes a housing; a tape (50) under tension to retract into the housing, at least one of the tape (50) and the housing are connected with the belt (16); a lens (56) configured to focus light on a pattern (52) that moves with the tape (50); and a photon detector (58) configured to detect light (57) reflected from the pattern (52). An optical decoder (26) determines movement of the belt (16) from the light reflected (57) from the pattern (52).

The present application relates to medical monitors for detecting a patient's respiration status. It finds particular application in improving the reliability and accuracy of detecting the respiration status of a patient undergoing a scanning procedure in a magnetic resonance (MRI) imaging environment and will be described with particular reference thereto.

MRI systems concentrate and direct relatively strong static magnetic fields (B₀), Radiofrequency fields (B₁), and magnetic field gradients (fast changing magnetic fields) to generate image data. Typically, electronic devices which operate inside or near the MRI scanner such as patient monitors are exposed to interference caused by these fields and gradients. The gradients and radio frequency field can also induce electrical currents which could harm the patient or the electronic devices.

During many MRI scanning procedures the patient's respiration is monitored while the patient is in the MRI environment. In breath hold imaging techniques, often physicians, technicians, and/or scanner electronics monitor patient respiration to determine when the patient is holding their breath in order to acquire image data at the correct time. The patient breathing can also be monitored during data acquisition to tag the acquired data with the corresponding respiratory state. Patient respiration is also monitored to inform medical personnel whether immediate clinical action is necessary.

Since the patient being monitored for respiration is located in the field of the MRI, the equipment must function properly when exposed to the fields and gradients mentioned above. One technique of monitoring patient respiration involves measuring respiration with an air bladder held around the patient's abdomen with a belt. As the patient breathes, the pressure in the bladder increases and decreases. A pressure transducer disposed in or adjacent the bladder converts the pressure differences to electrical signals. A problem with this is that the active electronic components are exposed to the interference produced by the MRI which causes potential accuracy, reliability, and safety issues. Another problem is that the active components can have an adverse effect on the MRI data and images. Another technique, involves sending a high-frequency, low amplitude voltage/current waveform through the ECG leads and performing a demodulation scheme where the variation in amplitude is extracted to derive a respiration waveform. A problem with this is that the leads used for MRI-compatible ECG are of high impedance which inhibits this technique from working without excessively high voltages/currents and could pose a patient risk. Moreover, the gradients can induce currents in the leads. Another technique uses remote cameras or lasers to monitor abdominal movement. However, the patient, technicians, equipment, or clinicians can block the laser light or the camera's view.

The present application provides a new and improved optical respiration status sensor which overcomes the above-referenced problems and others.

In accordance with one aspect, a subject monitor is provided. A belt circumscribes a cyclically moving portion of a subject, such as a chest cavity or abdomen. An optical sensing device includes a housing; a tape under tension to retract into the housing, where at least one of the tape and the housing are connected to the belt; a lens configured to focus light on a pattern that moves with the tape; and a photon detector configured to detect light reflected from the pattern. An optical decoder determines movement of the belt from the light reflected from the pattern.

In accordance with another aspect, an MR system is provided. An MR scanner generates MR data from a portion of a subject in an examination region. An optical sensing device in or adjacent the examination region includes a housing; a tape under tension to retract into the housing, where at least one of the tape and the housing are connected to the belt; a lens configured to focus light on a pattern that moves with the tape; and a photon detector configured to detect light reflected from the pattern. An optical decoder determines movement of the belt from the light reflected from the pattern.

In accordance with another aspect, a method for determining cyclic movement of subject is provided. Light is focused on a pattern that moves with a tape, the tape being connected to a belt that circumscribes a cyclically moving portion of a subject and is under tension to retract. The light reflected from the pattern is detected. The movement of the belt is detected from the light reflected from the pattern.

One advantage resides in the reliable detection of a patient's respiratory state.

Another advantage resides in interference free operation of a MRI scanner during a scanning procedure.

Other advantages reside in the accurate and reliable measurement of phase, rate, and other information concerning respiration or other cyclic anatomical motion. Still further advantages of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

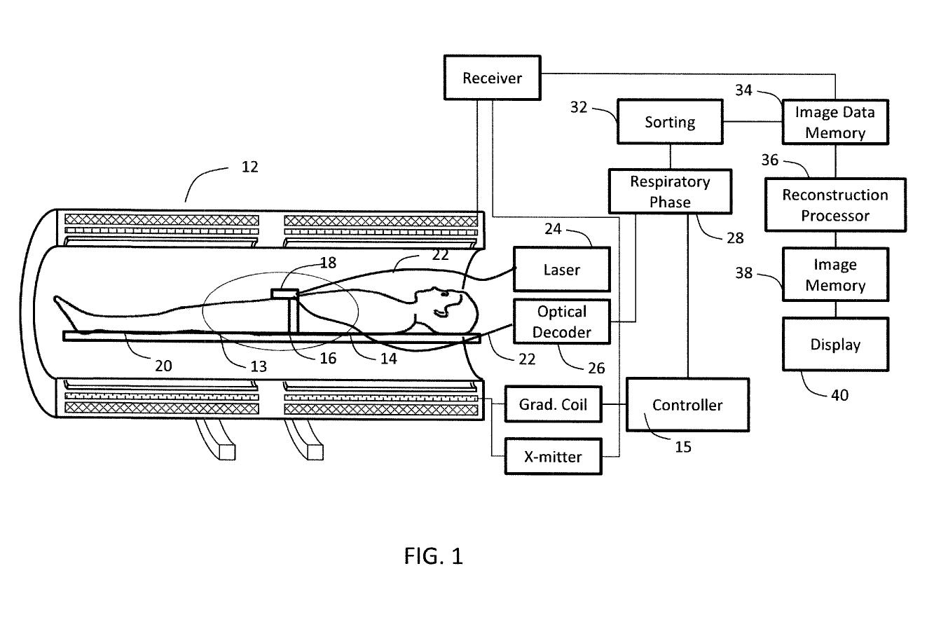

FIG. 1 is a diagrammatic illustration of an MRI and optical respiration status system in accordance with the present application.

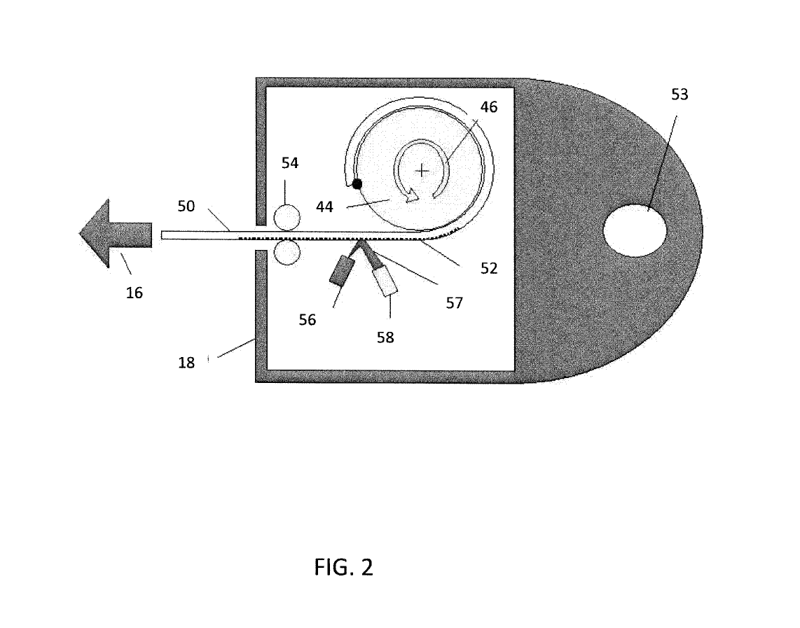

FIG. 2 is a diagrammatic illustration of one embodiment of an optical respiration status senor in according with the present application.

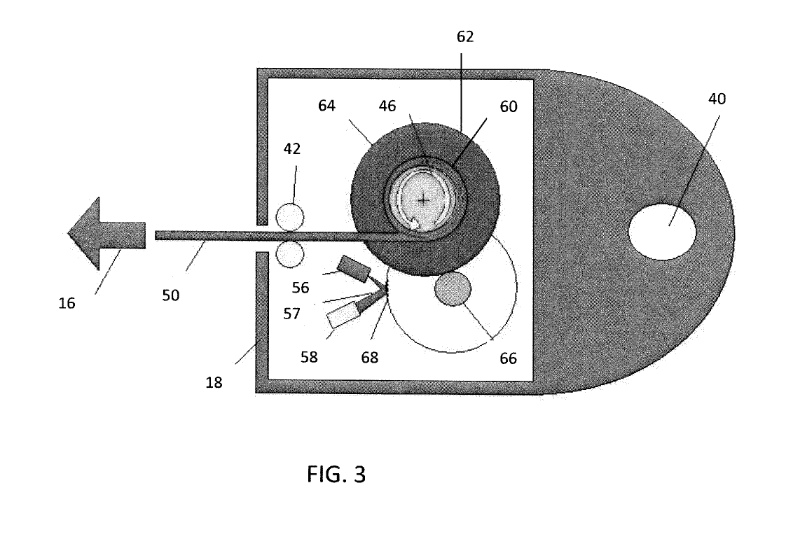

FIG. 3 is a diagrammatic illustration of another embodiment of an optical respiration status sensor in according with the present application.

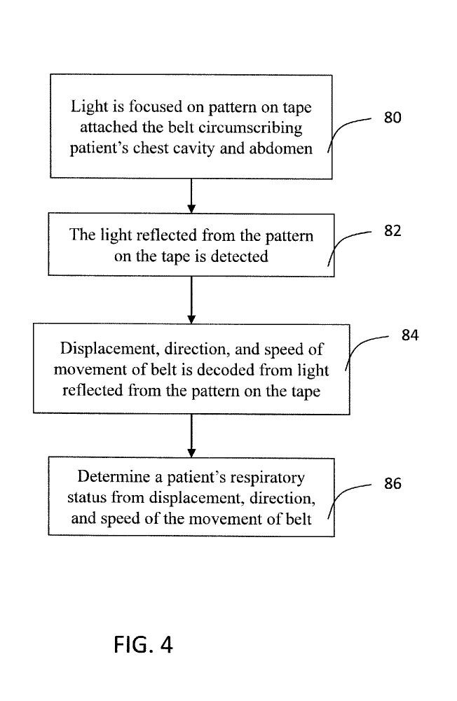

FIG. 4 is a flowchart diagram of the operation of an optical respiration status device.

With reference to FIG. 1, illustrated is a magnetic resonance (MR) imaging system 12 that includes a main magnet which generates a temporally uniform B₀ field through an examination region 13. The main magnet can be an annular or bore-type magnet, a C-shaped open magnet, other designs of open magnets, or the like. Gradient magnetic field coils disposed adjacent the main magnet serve to generate magnetic field gradients along selected axes relative to the B₀ magnetic field for spatially encoding magnetic resonance signals, for producing magnetization-spoiling field gradients, or the like. Typically, the gradient fields extend well beyond the imaging region and are typically strong fields a meter or more away.

A radio-frequency (RF) coil assembly, such as a whole-body radio frequency coil, is disposed adjacent the examination region 13. The RF coil assembly generates radio frequency B₁ pulses for exciting magnetic resonance in the aligned dipoles of the subject. The radio frequency coil assembly also serves to detect magnetic resonance signals emanating from the imaging region. Optionally, local, surface, head, or in vivo RF coils are provided in addition to or instead of the whole-body RF coil for more sensitive, localized spatial encoding, excitation, and reception of magnetic resonance signals. In a multi-element RF coil assembly, the RF coil assembly includes a plurality of individual coil elements to improve B₁ homogeneity and reduce specific absorption rate (SAR) in the subject.

To acquire magnetic resonance data of a patient 14, the portion of the patient 14 to be imaged is placed inside the examination region 13, preferably at or near an isocenter of the main magnetic field. A scan controller 15 controls gradient amplifiers which causes the gradient coils to apply the selected magnetic field gradient pulses across the imaging region, as may be appropriate to a selected magnetic resonance imaging or spectroscopy sequence. The scan controller controls an RF transmitter which causes the RF coil assembly to generate magnetic resonance excitation and manipulation B₁ pulses.

The scan controller also controls an RF receiver which is connected to the RF coil assembly to receive the generated magnetic resonance signals therefrom. In a multi-element RF coil assembly, the RF receiver typically includes a plurality of receivers or a single receiver with a plurality of receive channels, each receive channel includes a pre-amplifier operatively connected to a corresponding coil element of the coil assembly. In a single coil design, a single receive channel includes a single pre-amplifier which amplifies the received magnetic resonance signals.

A belt 16 circumscribes the patient's 14 chest cavity or abdomen. The belt 16 is attached to a optical sensing device 18 that senses the bi-directional movement of the belt 16 caused by the expansion and contraction of the patient's 14 chest cavity or abdomen caused by the patient's 14 breathing or other similar cyclic motion. The optical sensing device 18 is attached between ends of the belt 16 or between the belt 16 and a rigid point on a patient supports or bed 20 of the MRI 12 by a retaining loop or the like. The optical sensing device 18 is connected by one or more fiber optical cables 22 with a source 24 of light, such as a laser or light emitting diode, and an optical decoder 26. The light source 24 and the optical decoder 26 are positioned displaced from the examination region 13, preferably outside the 5 Gauss line or the imaging field of view. As the patient 14 breaths, cavity expansion causes the optical sensing device 18 to elongate and contract and to send optical signals indicative of the elongation and contraction. The optical decoder 26, located outside the examination region 13, produces electrical signals corresponding to the optical signals indicative of the elongation and contraction, e.g. pulses indicative of respiratory movement. A respiratory phase circuit or processor 28 converts the electrical signals indicative of respiratory movement into an indication of current respiratory phase, e.g. end exhale, full inhale, and various phases or states in between. In one embodiment, the respiratory phase circuit or processor 28 controls a display which displays the patient's 14 respiratory phase, respiratory rate, and other information concerning respiration or other cyclic anatomical motion.

The scan controller 15, in a breath hold embodiment, receives the respiratory phase, particularly the full inhale or other selected breath hold phase, from the respiratory phase circuit or processor 28. The scan controller 15 controls the MRI scanner 12 to acquire the data during the breath hold phase or other selected phases and pause in between. In another embodiment, image data from the receiver(s) of the MRI scanner 12 are sorted by a sorting routine or processor 32 in accordance with respiratory phase. An image data memory 34 stores the image data by phase. A reconstruction processor 36 reconstructs images in one or more selected phases which are stored in an image memory 38 and displayed on a display 40, sent to a central memory, or the like. For example, images can be reconstructed in a plurality of respiratory phases and displayed in cine fashion.

With reference to FIG. 2, in one embodiment of the optical sensing device 18 includes a tape 50 that carries a planar grid pattern or other recurring pattern 52, for example, orthogonally positioned grid lines of uniform spacing. The light source 24 provides light to illuminate at least a portion of the grid pattern. The optical decoder 26 receives the reflected light from an area at the plane of the grid pattern. The respiratory phase circuit or processor 28 interprets the displacement, direction, and/or speed of the elongation/contraction from the grid pattern on the tape 50 and correspondingly determines the respiratory phase of the patient. For example, the respiratory phase circuit or processor 28 interprets the detected light coming from the tape 50 as a series of bars where bars of widths 1, 2 and 3 cyclically reoccurring in a pattern. During expansion, the emitter would detect increasing pattern of bars e.g. 1,2,3,1,2,3 . . . . During contraction, the pattern moves the other way e.g. 3,2,1,3,2,1 . . . . The respiratory phase circuit or processor 28 recognizes the pattern and determines whether or not the patient is breathing based on the pattern movement. From this pattern movement, the respiratory phase circuit or processor 28 also determines the phase of the patient's respiratory cycle. Optionally, the respiratory phase circuit or processor 28 generates a breathing model for the patient.

While preparing the patient 14 for the MRI scan, the belt 16 is adjusted to circumscribe the patient's chest cavity or abdomen and attached to the tape 50 protruding from the optical sensing device 18. The optical sensing device 18 is attached to a rigid point on the bed 20, or the other end of the belt 16, by means of a retaining loop 53 or the like. The tape 50 passes through guides 54 located within the housing of the optical sensing device 18 and wraps around a spool 44. The spool 44 is mounted such that a torsion spring tension 46 or the like biases the tape 16 to wrap around the spool and retract into the optical sensing device 18. As the patient 14 breaths, cavity expansion pulls the tape out of the optical sensing device 18, and contraction allows the spool 44 to retract the tape 50 back into the optical sensing device 18. To detect this movement a lens 56 focuses the light conveyed by optic fiber 22 from the light source 24 onto the pattern 52 on the tape 50. Light 57 that reflects off the tape is received by a photon detector 58 and conveyed by the optic fiber 22 to the optical decoder 26. The pattern 52 on the tape 50 is configured such that the displacement and optionally the direction and speed of the moving tape can be interpreted by the respiratory phase circuit or processor 28, to extract the cardiac phase

With reference to FIG. 3, in another embodiment of the optical sensing device 18 gear reduction is used to increase the resolution and tension. The tape 50 passes through the guides 54 and wraps around a smaller outside diameter portion 60 of a stepped spool 62 with a larger outside diameter portion configured with gear teeth 64. This spool 62 is tensioned with a torsion spring 46 or the like to retract the tape, but with little resistance to expansion that occurs during breathing. The gear teeth 64 of the spool 62 drive a geared smaller diameter portion of a second stepped gear 66. A larger outside diameter of the second stepped gear 66 is imprinted with a series of patterns 68 that allow the respiratory phase circuit or processor 28 to determine displacement and preferably also direction and speed. By using the principles of change or reduction gears, the speed of the pattern 68 and the number of pattern markings per unit of belt movement is greatly increased. This improves the resolution. A lens 56 focuses light conveyed by optic fiber 22 from the light source 24 onto the pattern 68, and reflected light 57 is captured by a photon detector 58 and conveyed by the optic fiber 22 to the optical decoder 26. A case or enclosure of the optical sensing device 18 is constructed to keep out ambient light, and attach to a rigid point on the bed 20 or other end of the belt 16 by means of the retaining loop 40. The pattern 68 is configured such that the displacement, direction, and speed of the moving tape may be interpreted by the respiratory phase circuit or processor 28, such as a recurring pattern, sequence, or the like.

With reference to FIG. 4, illustrated is a flowchart of the optical respiration status sensor. In a step 80, the light is focused on the pattern on the tape or gear attached the belt circumscribing patient's chest cavity and abdomen. In a step 82, the light reflecting from the pattern is detected. In a step 84, the distance of displacement, and optionally the direction and the speed of the movement of the belt are decoded from the light reflected from the pattern on the tape. In a step 86, the patient's respiration phase is determined from the distance of displacement, direction and/or speed of the movement of the belt.

Although described in terms of respiration, other anatomical movement, particularly cyclic movement, can also be detected. For example, flexing of a muscle such as in the leg or arm can be determined Pulsing movement, such as from a spasm or possibly blood flow can be detected. Flexing a joint can be detected, such as by placing the belt over the calf and thigh to monitoring knee flexation.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A subject monitor comprising:
a belt that circumscribes a cyclically moving portion of a subject, such as a chest cavity or abdomen;
a optical sensing device including:
a housing;
a tape under tension to retract into the housing, at least one of the tape and the housing being connected with the belt;
a lens configured to focus light on a pattern that moves with the tape; and
a photon detector configured to detect light reflected from the pattern; and
a optical decoder which determines movement of the belt from the light reflected from the pattern.

2. The subject monitor according to claim 1, further including:
a respiration phase circuit or processor which determines a respiratory phase of the subject from the determined movement of the belt.

3. The subject monitor according to claim 1, wherein the optical sensing device and the subject are positioned inside an examination region of an MR scanner.

4. The subject monitor according to claim 1, wherein the optical sensing device is connected by one or more fiber optic cables with a light source and the optical decoder.

5. The subject monitor according to claim 1, wherein the light source and the optical decoder are displaced outside the MR scanner.

6. The subject monitor according to claim 1, wherein the pattern is configured to determine displacement, direction, and speed of the belt.

7. An MR system comprising:
an MR scanner which generates MR data from a portion of a subject in an examination region; and
the subject monitor according to claim 1 disposed the optical sensing device in or adjacent the examination region.

8. The MR system according to claim 7, further including:
a optical decoder displaced from the examination region and connected by one or more fiber optic cables to the optical sensing device.

9. The MR system according to claim 7, further including:
a light source displaced from the examination region and connected by one or more fiber optic cables to the optical sensing device.

10. The MR system according to claim 8, wherein the subject monitor is positioned inside a 5 Gauss line and the optical decoder is positioned outside the 5 Gauss line.

11. A method for determining cyclic movement of subject, the method comprising:
focusing light on a pattern that moves with a tape, the tape being connected to a belt that circumscribes a cyclically moving portion of a subject and under tension to retract;
detecting the light reflected from the pattern; and
determining the movement of the belt from the light reflected from the pattern.

12. The method according to claim 11, further including:
determining a respiratory phase of the subject from the movement of the belt.

13. The method according to claim 11, wherein an optical sensing device is disposed in the magnetic field.

14. The method according to claim 11, wherein the pattern is configured to determine displacement, direction, and speed of the belt.

15. The method according to claim 11, further including:
displaying at least one of the determine respiratory phase of the subject.
